(19) United States
(12) Patent Application Publication
(10) Pub. No.: US 2001/0031270 A1
(43) Pub. Date: Oct. 18, 2001

(54) Cosmetic compositions comprising an amphoteric starch and a cationic conditioner, and uses thereof

(76) Inventors: Veronique Douin, Paris (FR); Laurent Chesneau, Levallois Perret (FR); Sandrine Decoster, Saint Gratien (FR)

Correspondence Address: FINNEGAN, HENDERSON, FARABOW, GARRETT & DUNNER, L.L.P., 1300 I Street, N.W., Washington, DC, 20005-3315, US

(21) Appl. No.: 09/759,165

(22) Filed: Jan. 16, 2001

(30) Foreign Application Priority Data
Jan. 13, 2000 (FR) 00 00409

(51) Int. Cl. A61K 6/00; A61K 7/00; A61K 7/06; A61K 7/11; A61K 7/09; A61K 7/075; A61K 7/08

(52) U.S. Cl. 424/401; 424/70.1; 424/70.11; 424/70.12; 424/70.122; 424/70.13; 424/70.6; 424/70.5; 424/70.2; 424/70.17; 424/70.27; 424/70.28

(57) ABSTRACT

Cosmetic compositions comprising, in a cosmetically acceptable medium, at least one specific amphoteric starch and at least one cationic conditioner. This combination can provide cosmetic properties such as smoothness, lightness, and suppleness without certain cosmetic effects considered undesirable. These compositions can be used for washing and/or conditioning keratin materials such as the hair or the skin.

The present invention relates to novel cosmetic compositions comprising, in a cosmetically acceptable medium, at least one cationic conditioner and at least one amphoteric starch.

It is well known that hair that has been sensitized (i.e. damaged and/or embrittled) to varying degrees under the action of atmospheric agents or under the action of mechanical or chemical treatments, such as dyes, bleaches and/or permanent-waving, can be difficult to disentangle and to style, and may lack softness.

It has already been recommended to use conditioners, for example, cationic polymers or silicones, in compositions for washing or caring for keratin materials such as the hair, in order to facilitate the disentangling of the hair and to give it softness and suppleness. However, the cosmetic advantages mentioned above can be accompanied, on dried hair, by certain cosmetic effects considered undesirable, i.e., lankness of the hairstyle (lack of lightness of the hair), and lack of smoothness (hair not uniform from the root to the end).

In addition, the use of cationic polymers for this purpose may have various drawbacks. On account of their high affinity for the hair, some of these polymers can become deposited thereon to a large extent during repeated use, and may lead to adverse effects such as an unpleasant, laden feel, stiffening of the hair and interfiber adhesion which may effect styling. These drawbacks may be more accentuated in the case of fine hair, which lacks liveliness and body.

In summary, it is found that the current cosmetic compositions containing conditioners are not always entirely satisfactory.

The inventors have discovered that the combination of at least one amphoteric starch defined below with at least one certain cationic conditioner makes it possible to overcome at least one of these drawbacks.

Thus, after considerable research conducted in this matter, the inventors have found that by introducing at least one amphoteric starch into compositions, such as hair compositions based on conditioners, it is possible to limit, or even eliminate, at least one of the problems generally associated with the use of such compositions, i.e., for example, the lankness (charged feel following repeated applications) and the lack of smoothness and suppleness of the hair, while at the same time retaining at least one of the other advantageous cosmetic properties which are associated with conditioner-based compositions.

Moreover, when applied to the skin, for example in the form of a bubble bath or shower gel, the compositions of the invention can provide an improvement in the softness of the skin.

Thus, according to the present invention, a cosmetic composition is provided comprising, in a cosmetically acceptable medium, a) at least one amphoteric starch defined below, and b) at least one cationic conditioner chosen from polyquaternary ammonium polymers defined below, cationic silicones, quaternary ammonium salt surfactants, cyclopolymers of alkyldiallylamine, and cyclopolymers of dialkyldiallylammonium.

Another subject of the invention relates to the use of at least one amphoteric starch defined below in a cosmetic composition comprising at least one cationic conditioner chosen from polyquaternary ammonium polymers defined below, cationic silicones, quaternary ammonium salt surfactants, cyclopolymers of alkyldiallylamine, and cyclopolymers of dialkyldiallylammonium.

An additional subject of the invention relates to the use of at least one amphoteric starch defined below for the manufacture of a cosmetic composition comprising at least one cationic conditioner chosen from polyquaternary ammonium polymers defined below, cationic silicones, quaternary ammonium salt surfactants, cyclopolymers of alkyldiallylamine, and cyclopolymers of dialkyldiallylammonium.

Various subjects of the invention will now be described in detail. All of the meanings and definitions of the compounds used in the present invention given below are valid for all of the subjects of the invention.

As used herein, the term “conditioner” means any agent whose function is to improve at least one cosmetic property of the hair such as its softness, disentangling, feel, and static electricity.

The composition according to the invention comprises at least one amphoteric starch chosen from the compounds of the following formulae:

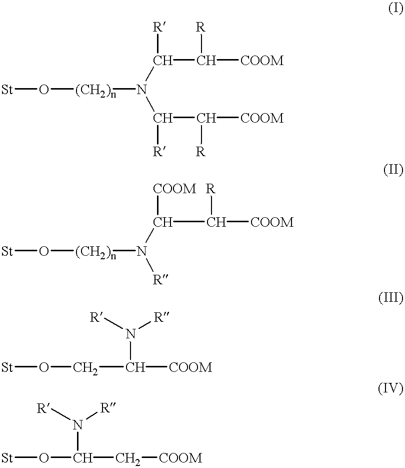

wherein:

St—O is a starch moiety;

R, which may be identical or different, are each chosen from a hydrogen atom and a methyl group;

R′, which may be identical or different, are each chosen from a hydrogen atom, a methyl group, and a —COOH group;

n is chosen from integers ranging from 2 to 3;

M, which may be identical or different, are each chosen from a hydrogen atom, an alkali metal, an alkaline-earth metal (such as Na, K, and Li), NH₄, quaternary ammonium to compounds, and organic amines; and

R″, which may be identical or different, are each chosen from a hydrogen atom and alkyl groups comprising from 1 to 18 carbon atoms.

These compounds are disclosed, for example, in U.S. Pat. Nos. 5,455,340 and 4,017,460, the disclosures of which are incorporated herein by reference.

The starch moieties may, for example, be derived from any plant sources of starch such as, for example, corn, potato, oat, rice, tapioca, sorghum, barley and wheat. The starch hydrolysates mentioned above may also be used. For example, in one embodiment the starch is derived from potato.

Certain embodiments of the present invention comprise the starches of formulae (I) and (II). Additional embodiments comprise starches modified with 2-chloroethylaminodipropionic acid, i.e., the starches of formulae (I) and (II) in which R, R′ and R″ represent a hydrogen atom and n is equal to 2.

The at least one amphoteric starch according to the invention can be used in the compositions in accordance with the present invention in amounts ranging for example from 0.01% to 10%, such as from 0.1% to 5% by weight, relative to the total weight of the composition.

The polyquaternary ammonium polymers are chosen from:

(1) diquaternary ammonium polymers comprising repeating units of formula:

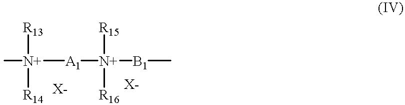

wherein:

R₁₃, R₁₄, R₁₅ and R₁₆, which may be identical or different, are each chosen from aliphatic groups comprising from 1 to 20 carbon atoms, alicyclic groups comprising from 1 to 20 carbon atoms, arylaliphatic groups comprising from 1 to 20 carbon atoms, lower hydroxyalkylaliphatic groups, and, additionally,

at least two of said R₁₃, R₁₄, R₁₅ and R₁₆, with the nitrogen atoms to which they are attached, form at least one heterocycle optionally comprising an additional heteroatom other than nitrogen, and, additionally,

R₁₃, R₁₄, R₁₅ and R₁₆, which may be identical or different, are each chosen from linear and branched C₁-C₆ alkyl groups substituted with at least one group chosen from nitrile groups, ester groups, acyl groups, amide groups and groups chosen from groups of formulae —CO—O—R₁₇—D and —CO—NH—R₁₇—D wherein R₁₇ is chosen from alkylene groups and D is chosen from quaternary ammonium groups;

A₁ and B₁, which may be identical or different, are each chosen from polymethylene groups comprising from 2 to 20 carbon atoms, chosen from linear and branched, saturated and unsaturated polymethylene groups, wherein said polymethylene groups may optionally comprise, optionally linked to and optionally intercalated in the main chain, at least one entity chosen from aromatic rings, oxygen atoms, sulfur atoms, sulfoxide groups, sulfone groups, disulfide groups, amino groups, alkylamino groups, hydroxyl groups, quaternary ammonium groups, ureido groups, amide groups and ester groups; and

X⁻ is an anion chosen from anions derived from inorganic acids and anions derived from organic acids; and

A₁, R₁₃ and R₁₅ may optionally form, together with the two nitrogen atoms to which they are attached, at least one piperazine ring;

with the proviso that if A₁ is chosen from linear and branched, saturated and unsaturated alkylene groups and linear and branched, saturated and unsaturated hydroxyalkylene groups, B₁ may also be chosen from groups of formula:

(CH₂)ₙ—CO—D—OC—(CH₂)ₙ—

wherein D is chosen from:

a) glycol residues of formula: —O—Z—O—, wherein Z is chosen from linear and branched hydrocarbon groups and groups chosen from groups of formulae:

—(CH₂—CH₂—O)ₓ—CH₂—CH₂—;

and

—[CH₂—CH(CH₃)—O]_{y}—CH₂—CH(CH₃)—

wherein x and y, which may be identical or different, are each chosen from integers ranging from 1 to 4 (in which case x and y represent a defined and unique degree of polymerization) and any number ranging from 1 to 4 (in which case x and y represent an average degree of polymerization);

b) bis-secondary diamine residues such as piperazine derivatives;

c) bis-primary diamine residues chosen from residues of formula: —NH—Y—NH—, wherein Y is chosen from linear and branched hydrocarbon groups and residues of formula —CH₂—CH₂—S—S—CH₂—CH₂—; and

d) ureylene groups of formula: —NH—CO—NH—.

In one embodiment, X⁻ is an anion chosen from chloride atoms and bromide atoms.

According to the present invention, the quarternary diammonium polymers have a number-average molecular mass ranging for example from 1000 to 100,000.

For example, polymers of this type are described in French Patent Nos. 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020, the disclosures of which are incorporated herein by reference.

Further, according to the present invention, polymers comprising repeating units of formula (VI) may be used:

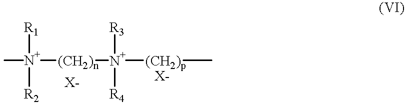

wherein:

R₁, R₂, R₃ and R₄, which may be identical or different, are each chosen from alkyl groups comprising from 1 to 4 carbon atoms, and hydroxyalkyl groups comprising from 1 to 4 carbon atoms;

n and p, which may be identical or different, are each chosen from integers ranging from 2 to 20; and

X⁻ is an anion chosen from anions derived from inorganic acids and anoins derived from organic acids.

In one embodiment, R₁, R₂, R₃ and R₄ are each a methyl group, n=3, p=6 and X=Cl. This unit is commonly known as hexadimethrine chloride according to INCI (CTFA) nomenclature.

(2) polyquaternary ammonium polymers comprising at least one unit of formula (VII):

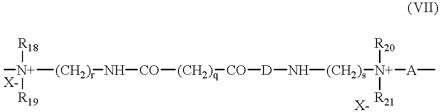

wherein:

R₁₈, R₁₉, R₂₀ and R₂₁, which may be identical or different, are each chosen from a hydrogen atom, a methyl group, an ethyl group, a propyl group, a β-hydroxyethyl group, a β-hydroxypropyl group, and a —CH₂CH₂(OCH₂CH₂)ₚOH group, wherein p is an integer ranging from 0 to 6,

with the proviso that R₁₈, R₁₉, R₂₀ and R₂₁ are all not simultaneously a hydrogen atom;

r and s, which may be identical or different, are each chosen from an integer ranging from 1 to 6;

q is an integer ranging from 1 to 34;

X⁻ is chosen from anions of inorganic and organic acids, such as a halide,

D is chosen from direct bonds and —(CH₂)ₜ—CO— groups, wherein t is 4 or 7,

A is chosen from dihalide groups, and a group of formula —CH₂—CH₂—O—CH₂—CH₂—.

For example, such compounds are described in patent application EP-A-122,324, the disclosure of which is incorporated by reference.

Non-limiting examples of the polyquarternary ammonium polymers are “Mirapol A 15”, “Mirapol AD1”, “Mirapol AZ1” and “Mirapol 175” sold by the company Miranol.

According to the present invention, the term “cationic silicone” denotes any silicone comprising at least one group chosen from primary amine groups, secondary amine groups, tertiary amine groups and quaternary ammonium groups. Non-limiting examples include:

(a) the polysiloxanes referred to in the CTFA dictionary as “amodimethicone” of formula:

m and n, which may be identical or different, are numbers such that the sum (n+m) can range from 1 to 2,000, such as from 50 to 150, wherein n may denote a number ranging from 0 to 1,999, such as from 49 to 149, and m may denote a number ranging from 1 to 2,000, such as from 1 to 10;

R′, which may be identical or different, are each chosen from monovalent groups of formula —C_{q}H_{2q}L in which q is a number ranging from 2 to 8 and L is an optionally quaternized amine group chosen from the groups:

—NR″—CH₂—CH₂—N′(R″)₂,

—N(R″)₂,

—N^{⊕}(R″)₃A⁻,

—N^{⊕}(R″)₂A⁻,

—NH₂^{⊕}(R″)A⁻,

and

—N(R″)—CH₂—CH₂—N^{⊕}R″H₂A⁻,

in which:

R″, which may be identical or different, are each chosen from a hydrogen atom, a phenyl group, a benzyl group, and saturated monovalent hydrocarbon groups, such as an alkyl group comprising from 1 to 20 carbon atoms, and

A⁻ is a halide ion such as, for example, an ion chosen from fluoride, chloride, bromide and iodide ions.

A product corresponding to this definition is the silicone known as “trimethylsilylamodimethicone”, of formula:

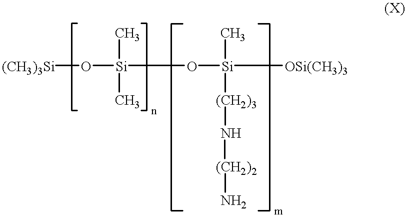

wherein:

n and m have the meanings given above.

Such polymers are described for example in patent application EP-A-95238, the disclosure of which is incorporated herein by reference.

(c) aminosilicones of formula:

[chemical structure]

wherein:

R₅, which may be identical or different, are each chosen from monovalent hydrocarbon groups comprising from 1 to 18 carbon atoms, such as a group chosen from C₁-C₁₈ alkyl groups and C₂-C₁₈ alkenyl groups, for example a methyl group;

R₆ is a divalent hydrocarbon group, such as a group chosen from C₁-C₁₈ alkylene groups and divalent C₁-C₁₈ groups, for example a C₁-C₁₈, alkylenoxy group connected to the Si by an SiC bond;

Q⁻ is chosen from anions such as halide ions, for example chloride ions, and organic acid salts (acetate, etc.);

r is an average statistical value ranging from 2 to 20, such as from 2 to 8;

s is an average statistical value ranging from 20 to 200, such as from 20 to 50.

Such aminosilicones are described in U.S. Pat. No. 4,185,087, the disclosure of which is incorporated herein by reference.

A silicone which falls within this class is the silicone sold by the company Union Carbide under the name “Ucar Silicone ALE 56”.

d) quaternary ammonium silicones of formula:

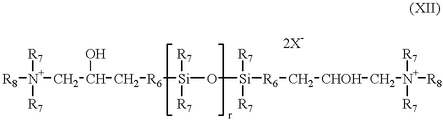

wherein:

R₇, which may be identical or different, are each chosen from monovalent hydrocarbon groups comprising from 1 to 18 carbon atoms, such as a C₁-C₁₈ alkyl groups, C₂-C₁₈ alkenyl groups, and rings comprising 5 to 6 carbon atoms. For example, R₇ can be a methyl group;

R₆, which may be identical or different, are each chosen from divalent hydrocarbon groups, such as C₁-C₁₈ alkylene groups and divalent C₁-C₁₈ groups, for example a C₁-C₈, alkylenoxy group connected to the Si by an SiC bond;

R₈, which may be identical or different, are each chosen from a hydrogen atom, monovalent hydrocarbon groups comprising 1 to 18 carbon atoms, such as C₁-C₁₈ alkyl groups, C₂-C₁₈ alkenyl groups and groups of formula —R₆—NHCOR₇;

X⁻ is chosen from anions such as halide ions, for example chloride ions, and organic acid salts (acetate, etc.);

r is an average statistical value ranging from 2 to 200, such as from 5 to 100.

These silicones are described, for example, in application EP-A-0,530,974, the disclosure of which is incorporated herein by reference.

Other non-limiting examples of these silicones are silicones sold by the company Goldschmidt under the names Abil Quat 3270, Abil Quat 3272 and Abil Quat 3474.

e) aminosilicones of formula (XIII):

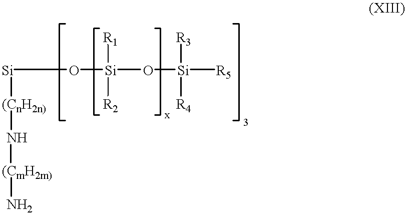

wherein:

R₁, R₂, R₃ and R₄, which may be identical or different, are each chosen from C₁-C₄ alkyl groups and a phenyl group,

R₅ is chosen from C₁-C₄ alkyl groups and a hydroxyl group,

n is an integer ranging from 1 to 5,

m is an integer ranging from 1 to 5, and

x is chosen such that the amine number ranges for example from 0.01 to 1 meq/g.

According to the present invention, the aminosilicones can be in the form of at least one composition chosen from oils, aqueous solutions, alcoholic solutions, and aqueous-alcoholic solutions, in the form of dispersions and emulsions.

In one embodiment of the present invention, for example, the aminosilicones can be in the form of emulsions, such as in the form of microemulsions and nanoemulsions.

For example, the product sold under the name “Cationic Emulsion DC 929” by the company Dow Corning, which comprises, besides amodimethicone, a cationic surfactant derived from tallow fatty acids, referred to as tallowtrimonium (CTFA), in combination with a nonionic surfactant, known under the name “Nonoxynol 10”, can be used.

In another example, the product sold under the name “Catonic Emulsion DC 939” by the company Dow Corning, which comprises, besides amodimethicone, a cationic surfactant, trimethylcetylammonium chloride, in combination with a nonionic surfactant, trideceth-12, can be used.

Another commercial product which can be used according to the present invention is the product sold under the name “Dow Corning Q2 7224” by the company Dow Corning, comprising the trimethylsilylamodimethicone of formula (X), a nonionic surfactant of formula: C₈H₁₇—C₆H₄—(OCH₂CH₂)ₙ—OH in which n=40, also known as octoxynol-40, another nonionic surfactant of formula: C₁₂H₂₅—(OCH₂—CH₂)ₙ—OH in which n=6, also known as isolaureth-6, and glycol.

The cationic quaternary ammonium salt surfactants according to the present invention may, for example, be chosen from:

A) quaternary ammonium salts of formula (XIV) below:

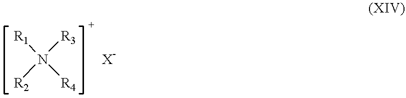

in which:

the radicals R₁, R₂, R₃, and R₄, which may be identical or different, are independently chosen from linear and branched aliphatic radicals comprising from 1 to 30 carbon atoms, and aromatic radicals, such as C₆-C₂₀ aromatic radicals (for example, aryl and alkylaryl), wherein the aliphatic radicals can comprise hetero atoms such as, oxygen, nitrogen, sulfur and halogens, and wherein the aliphatic radicals are chosen, for example, from alkyl, alkoxy, polyoxy(C₂-C₆)alkylene, alkylamide, (C₁₂-C₂₂)alkylamido(C₂-C₆)alkyl, (C₁₂-C₂₂)alkylacetate and hydroxyalkyl radicals, comprising from 1 to 30 carbon atoms;

X⁻ is an anion chosen from halides, phosphates, anions derived from organic acids, (C₂-C₆)alkyl sulfates, alkyl sulfonates, and alkylaryl sulfonates.

The compounds of formula (XIV) can be chosen from, for example, (a) compounds comprising at least two fatty aliphatic radicals comprising from 8 to 30 carbon atoms, (b) compounds comprising at least one fatty aliphatic radical comprising from 17 to 30 carbon atoms, and (c) compounds comprising at least one aromatic radical.

B) Quaternary ammonium salts of imidazolinium, such as, for example, the salts of formula (XV) below:

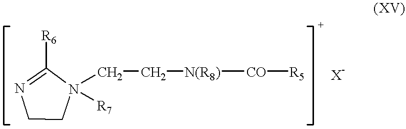

in which:

R₅ is chosen from alkenyl and alkyl radicals comprising from 8 to 30 carbon atoms, for example radicals derived from tallow fatty acid,

R₆ is chosen from a hydrogen atom, C₁-C₄ alkyl radicals, and alkenyl and alkyl radicals comprising from 8 to 30 carbon atoms,

R₇ is chosen from C₁-C₄ alkyl radicals,

R₈ is chosen from a hydrogen atom and C₁-C₄ alkyl radicals,

X⁻ is an anion chosen from halides, phosphates, acetates, lactates, alkyl sulfates, alkyl sulfonates, and alkylaryl sulfonates.

For example, R₅ and R₆, which may be identical or different, are independently chosen from alkenyl and alkyl radicals comprising from 12 to 21 carbon atoms, for example, radicals derived from tallow fatty acid, R₇ is methyl, and R₈ is hydrogen.

Such products are, for example, (1) Quaternium-27 (International Cosmetic Ingredient Dictionary and Handbook, hereafter “CTFA”, 1997), i.e., “Rewoquat” W75, W75PG, and W90, and (2) Quaternium-83 (CTFA 1997), i.e., “Rewoquat” W75HPG, which are sold by the company Witco.

C) Diquaternary ammonium salts of formula (XVI):

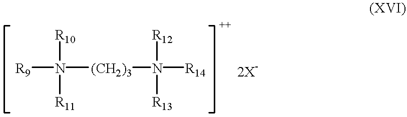

in which:

R₉ is chosen from aliphatic radicals comprising from 16 to 30 carbon atoms,

R₁₀, R₁₁, R₁₂, R₁₃ and R₁₄, which may be identical or different, are independently chosen from a hydrogen atom and alkyl radicals comprising from 1 to 4 carbon atoms, and

X⁻ is an anion chosen from halides, acetates, phosphates, nitrates and methyl sulfates.

For example, such diquaternary ammonium salts can comprise propane tallow diammonium dichloride.

D) Quaternary ammonium salts comprising at least one ester function. The quaternary ammonium salts comprising at least one ester function that can be used according to the present invention are, for example, those of formula (XVII) below:

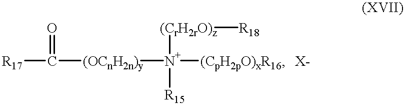

in which:

R₁₅ is chosen from C₁-C₆ alkyl radicals and C₁-C₆ hydroxyalkyl and C₁-C₆ dihydroxyalkyl radicals;

R₁₆ is chosen from:

(i) acyl groups of the following formula:

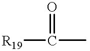

wherein R₁₉ is defined below,

(ii) linear and branched, saturated and unsaturated, C₁-C₂₂ hydrocarbon-based radicals, and

(iii) a hydrogen atom;

R₁₈ is chosen from:

(i) acyl groups of the following formula:

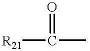

wherein R₂₁ is defined below,

ii) linear and branched, saturated and unsaturated, C₁-C₆ hydrocarbon-based radicals, and

(iii) a hydrogen atom;

R₁₇, R₁₉ and R₂₁, which may be identical or different, are independently chosen from linear and branched, saturated and unsaturated, C₇-C₂₁ hydrocarbon-based radicals;

n, p and r, which may be identical or different, are independently chosen from integers ranging from 2 to 6;

y is an integer ranging from 1 to 10;

x and z, which may be identical or different, are independently chosen from integers ranging from 0 to 10;

X⁻ is chosen from simple and complex, organic and inorganic anions;

provided that the sum x+y+z is from 1 to 15, and that when x is 0, then R₁₆ is chosen from linear and branched, saturated and unsaturated, C₁-C₂₂ hydrocarbon-based radicals, and that when z is 0, then R₁₈ is chosen from linear and branched, saturated and unsaturated, C₁-C₆ hydrocarbon-based radicals.

In one embodiment of the present invention, the R₁₅ alkyl radicals may be linear and branched and further, for example, linear.

For example, R₁₅ may be chosen from a methyl group, an ethyl group, a hydroxyethyl group, and a dihydroxypropyl radical, and further for example from a methyl group and an ethyl group.

The sum x+y+z may for example range from 1 to 10.

When R₁₆ is chosen from linear and branched, saturated and unsaturated, C₁-C₂₂ hydrocarbon-based radicals, R₁₆ may be long and comprise from 12 to 22 carbon atoms, or short and comprise from 1 to 3 carbon atoms.

When R₁₈ is chosen from linear and branched, saturated and unsaturated, C₁-C₆ hydrocarbon-based radicals, R₁₈ may for example comprise from 1 to 3 carbon atoms.

R₁₇, R₁₉ and R₂₁, which may be identical or different, can, for example, be independently chosen from linear and branched, saturated and unsaturated C₁₁-C₂₁ hydrocarbon-based radicals, and for example from linear and branched, saturated and unsaturated, C₁₁-C₂₁ alkyl and alkenyl radicals.

x and z, which may be identical or different, can for example independently be chosen from 0 or 1.

y for example may be equal to 1.

n, p and r, which may be identical or different, can for example be independently chosen from 2 and 3 and in one embodiment equal to 2.

The anion for example can be chosen from halides (chloride, bromide, and iodide) and alkyl sulfates, such as methyl sulfate. However, methanesulfonate, phosphate, nitrate, tosylate, anions derived from organic acids, such as acetate and lactate, and any other anions compatible with the ammonium comprising an ester function, may be used.

As a further example, the anion X⁻ can be chosen from chloride and methyl sulfate.

Further examples of ammonium salts of formula (XVII) are those in which:

R₁₅ is chosen from a methyl group and an ethyl group,

x and y are equal to 1;

z is equal to 0 or 1;

n, p and r are equal to 2;

R₁₆ is chosen from:

(i) acyl groups

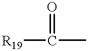

wherein R₁₉ is defined below,

(ii) a methyl group, an ethyl group, and C₁₄-C₂₂ hydrocarbon-based groups, and

(iii) a hydrogen atom;

R₁₈ is chosen from:

(i) acyl groups

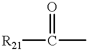

wherein R₂₁ is defined below,

(ii) a hydrogen atom;

R₁₇, R₁₉, and R₂₁, which may be identical or different, are independently chosen from linear and branched, saturated and unsaturated, C₁₃-C₁₇ hydrocarbon-based radicals, such as from linear and branched, saturated and unsaturated C₁₃-C₁₇ alkyl and alkenyl radicals.

The hydrocarbon-based radicals can for example be linear.

Representative compounds of formula (XVII) are chosen from diacyloxyethyl-dimethylammonium, diacyloxyethylhyd roxyethylmethylammonium, monoacyloxyethyldihydroxyethylmethylammonium, triacyloxyethylmethylammonium and monoacyloxyethylhydroxyethyldimethylammonium salts (for example chloride and methyl sulfate). The acyl radicals can comprise for example from 14 to 18 carbon atoms and can for example be obtained from plant oils, such as palm oil and sunflower oil. When the compound comprises several acyl radicals, these radicals, which may be independently chosen, may independently be identical or different.

These products are obtained, for example, by direct esterification of compounds chosen from triethanolamine, triisopropanolamine, alkyldiethanolamines and alkyldiisopropanolamines, which are optionally oxyalkylenated, with fatty acids or with fatty acid mixtures of plant or animal origin, and by transesterification of the methyl esters thereof. This esterification is followed by a quaternization using an alkylating agent such as alkyl halides (such ad methyl and ethyl halides), dialkyl sulfates (for example dimethyl and diethyl sulfates), methyl methanesulfonate, methyl para-toluenesulfonate, glycol chlorohydrin and glycerol chlorohydrin.

Such compounds are sold, for example, under the names Dehyquart by the company Henkel, Stepanquat by the company Stepan, Noxamium by the company Ceca and Rewoquat WE 18 by the company Rewo-Witco.

It is also possible to use the ammonium salts comprising at least one ester function, described in U.S. Pat. Nos. 4,874,554 and 4,137,180, the disclosures of which are incorporated by reference herein.

Representative quaternary ammonium salts of formula (XIV) include tetraalkylammonium chlorides such as, for example, dialkyldimethylammonium chlorides and alkyltrimethylammonium chlorides, in which the alkyl radical comprises from 12 to 22 carbon atoms, for example behenyltrimethylammonium chloride, distearyldimethylammonium chloride, cetyltrimethylammonium chloride, and benzyldimethylstearylammonium chloride, and, stearamidopropyidimethyl(myristyl acetate)ammonium chloride sold under the name “Cepharyl 70” by the company Van Dyk.

Cyclopolymers of alkyldiallylamine and cyclopolymers of dialkyldiallylammonium, such as homopolymers and copolymers comprising, as the main constituent of the chain, at least one unit chosen from units of formulae (XVIII) and (XIX):

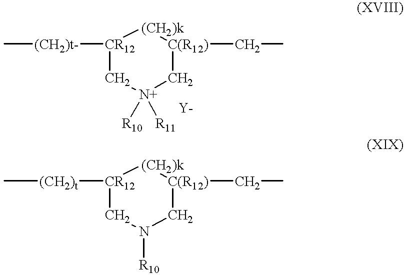

wherein:

k and t, which may be identical or different, are each chosen from 0 and 1, with the proviso that the sum of k +t is equal to 1;

R₁₂, which may be identical or different, are each chosen from a hydrogen atom and a methyl group;

R₁₀ and R₁₁, which may be identical or different, are each chosen from alkyl groups comprising from 1 to 22 carbon atoms, hydroxyalkyl groups wherein the alkyl group optionally comprises from 1 to 5 carbon atoms, lower C₁-C₄ amidoalkyl groups, and, additionally,

R₁₀ and R₁₁, together with the nitrogen atom to which they are commonly attached, form at least one heterocyclic group, such as piperidyl groups and morpholinyl groups;

Y⁻ is an anion, such as bromide, chloride, acetate, borate, citrate, tartrate, bisulfate, bisulfite, sulfate and phosphate. For example, such polymers are described in French patent 2,080,759 and in its Certificate of Addition 2,190,406, the disclosures of which are incorporated herein by reference.

In one embodiment of the present invention, R₁₀ and R₁₁, which may be identical or different, are each chosen from alkyl groups comprising from 1 to 4 carbon atoms.

Non-limiting examples of the polymers defined above include the dimethyldiallylammonium chloride homopolymer sold under the name “Merquat 100” by the company Calgon (and its homologues of low weight-average molecular mass) and copolymers of diallyldimethylammonium chloride and of acrylamide, sold under the name “Merquat 550”.

In certain embodiments of the present invention, combinations of conditioners are used.

According to the present invention, the at least one cationic conditioner may be present in amounts ranging for example from 0.001% to 10% by weight, such as from 0.01% to 5% by weight, and further such as from 0.1% to 3% by weight, relative to the total weight of the final composition.

The compositions of the present invention can also comprise at least one surfactant chosen from anionic, amphoteric and nonionic surfactants, which is present in an amount ranging for example from 0.1% to 60% by weight relative to the total weight of the composition, such as from 3% to 40%, and further such as from 5% to 30%.

The at least one surfactant chosen from anionic, amphoteric and nonionic surfactants, which is suitable for carrying out the present invention can, for example, include the following:

(i) Anionic Surfactant(s):

Representative anionic surfactants include salts (for example alkaline salts, such as sodium salts, ammonium salts, amine salts, amino alcohol salts and magnesium salts) of the following compounds: alkyl sulfates, alkyl ether sulfates, alkylamidoether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates. The alkyl and acyl radicals of all of these various compounds can for example comprise from 8 to 24 carbon atoms, and the aryl radicals can for example be chosen from phenyl and benzyl groups.

For example, anionic surfactants can be chosen from fatty acid salts such as the salts of oleic, ricinoleic, palmitic and stearic acids, coconut oil acid and hydrogenated coconut oil acid and acyl lactylates in which the acyl radical comprises from 8 to 20 carbon atoms. At least one weakly anionic surfactant can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, for example, those comprising from 2 to 50 ethylene oxide groups.

As a further example, the anionic surfactant can be at least one salt chosen from alkyl sulfate salts and alkyl ether sulfate salts.

(ii) Nonionic Surfactant(s):

Useful nonionic surfactants include compounds that are well known per se (see for example in this respect “Handbook of Surfactants” by M. R. Porter, published by Blackie & Son (Glasgow and London), 1991, pp. 116-178), the disclosure of which is incorporated by reference herein. Thus, nonionic surfactants can include polyethoxylated, polypropoxylated and polyglycerolated fatty acids, alkylphenols, α-diols and alcohols having a fatty aliphatic chain comprising, for example, 8 to 18 carbon atoms, it being possible for the number of ethylene oxide and propylene oxide groups to range for example from 2 to 50 and for the number of glycerol groups to range for example from 2 to 30. Mention may also be made of copolymers of ethylene oxide and of propylene oxide, condensates of ethylene oxide and of propylene oxide with fatty alcohols; polyethoxylated fatty amides for example comprising from 2 to 30 mol of ethylene oxide, polyglycerolated fatty amides comprising on average 1 to 5, such as from 1.5 to 4, glycerol groups; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides and N-acylaminopropylmorpholine oxides.

(iii) Amphoteric Surfactant(s):

Representative amphoteric surfactants include surfactants chosen from aliphatic secondary and aliphatic tertiary amine derivatives in which the aliphatic radical is chosen from linear and branched chain radicals comprising 8 to 22 carbon atoms and comprising at least one water-soluble anionic group (chosen for example from carboxylate, sulfonate, sulfate, phosphate and phosphonate); mention may also be made of (C₈-C₂₀)alkylbetaines, sulfobetaines, (C₈-C₂₀)alkylamido(C₁-C₆)alkyl betaines and (C₈-C₂₀)alkylamido(C₁-C₆)alkylsulfobetaines.

Representative amine derivatives include the products sold under the name Miranol, as described in U.S. Pat. Nos. 2,528,378 and 2,781,354, the disclosures of which are incorporated by reference herein, and having the structures:

R₂—CONHCH₂CH₂—N⁺(R₃)(R₄)(CH₂COO—) (2)

in which:

R₂ is chosen from alkyl radicals derived from an acid R₂—COOH present in hydrolysed coconut oil, heptyl, nonyl and undecyl radicals,

R₃ is chosen from a P-hydroxyethyl group, and

R₄ is chosen from a carboxymethyl group;

and

R₅—CONHCH₂CH₂—N(B)(C) (3)

in which:

(B) is —CH₂CH₂OX′, with X′ chosen from a —CH₂CH₂—COOH group and a hydrogen atom,

(C) is —(CH₂)_{z}—Y′, with z=1 or 2, and with Y′ chosen from —COOH and —CH₂—CHOH—SO₃H radicals,

R₅ is chosen from alkyl radicals, such as (a) alkyl radicals of an acid R₅—COOH present in oils chosen from coconut oil and hydrolysed linseed oil, (b) alkyl radicals, such as C₇, C₈, C₁₁ and C₁₃ alkyl radicals, and (c) C₁₇ alkyl radicals and the iso forms, and unsaturated C₁₇ radicals.

Such representative compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

By way of example, mention may be made of the cocoamphodiacetate sold under the trade name Miranol C2M Concentrate by the company Rhône-Poulenc.

In the compositions in accordance with the present invention, at least two surfactants of different types can be used. Representative compositions include compositions comprising (a) more than one anionic surfactant, (b) at least one anionic surfactant and at least one amphoteric surfactant, and (c) at least one anionic surfactant and at least one nonionic surfactant. In one embodiment, the composition can comprise at least one anionic surfactant and at least one amphoteric surfactant.

The at least one anionic surfactant used for example, can be chosen from (C₁₂-C₁₄)alkyl sulfates of sodium, of triethanolamine and of ammonium; (C₁₂-C₁₄)alkyl ether sulfates of sodium, of triethanolamine and of ammonium, oxyethylenated with 2.2 mol of ethylene oxide; sodium cocoyl isethionate; and sodium (C₁₄-C₁₆)-α-olefin sulfonate, and used in combination with an amphoteric surfactant chosen from:

amphoteric surfactants such as the amine derivatives known as disodium cocoamphodipropionate and sodium cocoamphopropionate, sold for example by the company Rhône-Poulenc under the trade name “Miranol C2M Conc®” as an aqueous solution comprising 38% active material, and under the name Miranol C32; and

amphoteric surfactants of zwitterionic type, such as alkylbetaines, for example the cocobetaine sold under the name “Dehyton AB 30” as an aqueous solution comprising 32% AM by the company Henkel.

The composition of the present invention may also comprise at least one additive chosen from thickeners (chosen from associative and non-associative thickeners), fragrances, nacreous agents, preserving agents, silicone sunscreens, non-silicone sunscreens, vitamins, provitamins, cationic polymers (in addition to cationic polymers disclosed above), amphoteric polymers, anionic polymers, nonionic polymers, proteins, protein hydrolysates, 18-methyleicosanoic acid, hydroxy acids, panthenol, volatile silicones, non-volatile silicones, cyclic silicones, linear silicones, crosslinked silicones, modified silicones and non-modified silicones, and any other additive conventionally used in cosmetics which does not affect the properties of the compositions according to the invention.

These additives may be present in the composition according to the present invention in amounts ranging for example from 0% to 20% by weight relative to the total weight of the composition. The amount of each additive can be determined by those skilled in the art depending on its nature and its function.

The compositions according to the present invention have a final pH ranging for example from 2 to 10, such as from 3 to 6.5. The pH can be adjusted to the desired value by adding at least one base (chosen from organic and inorganic bases) to the composition, such as bases chosen from aqueous ammonia and primary, secondary and tertiary (poly)amines for example monoethanolamine, diethanolamine, triethanolamine, isopropanolamine and 1,3-propanediamine. The pH can also be adjusted to the desired value by adding at least one acid, such as a carboxylic acid such as, for example, citric acid.

The compositions in accordance with the present invention may be used for washing and treating keratin materials such as the hair, the skin, the eyelashes, the eyebrows, the nails, the lips and the scalp, for example, the hair.

The compositions according to the present invention may comprise rinse-out and leave-in conditioner compositions.

The compositions according to the present invention may further comprise detergent compositions such as shampoos, shower gels and bubble baths and may also include make-up-removing products. In these embodiments of the present invention, the compositions comprise a washing base comprising at least one surfactant, wherein the washing base is generally aqueous.

The at least one surfactant optionally comprising the washing base may be chosen from the anionic, amphoteric and nonionic surfactants as defined above.

The quantity and quality of the washing base affords the final composition satisfactory foaming and detergent power.

The washing base may be present in an amount ranging for example from 4% to 50% by weight, such as from 6% to 35% by weight, and further such as from 8% to 25% by weight, relative to the total weight of the final composition.

Another subject of the present invention is a process for treating keratin materials such as the skin and the hair, which comprises applying a cosmetic composition as defined above to the keratin materials and then optionally rinsing it out with water.

This process according to the present invention allows, for example, maintenance of the hairstyle and treatment of, care of, washing of, and removal of make-up from the skin, the hair and any other keratin material.

The compositions of the present invention may also be in the form of at least one composition chosen, for example, from permanent-waving, straightening, dyeing and bleaching compositions. The compositions of the present invention may also be in the form of at least one rinse-out composition which may be applied before and after dyeing, and during bleaching, permanent-waving and straightening the hair. The compositions of the present invention may also be applied between steps of permanent-waving and hair-straightening operations.

The compositions according to the present invention may also be in the form of at least one lotion chosen from aqueous and aqueous-alcoholic lotions for skin care and hair care.

The cosmetic compositions according to the present invention may, for example, be in the form of at least one composition chosen from gels, milks, creams, emulsions, thickened lotions and mousses, and may, for example, be used for the skin, the nails, the eyelashes, the lips and the hair.

The compositions may be packaged in various forms, such as in vaporizers, pump-dispenser bottles and in aerosol containers in order to, for example, ensure application of the composition in vaporized form or in the form of a mousse. Such packaging forms are indicated, for example, when it is desired to obtain a spray, a lacquer or a mousse for treating the hair.

In all of the text hereinabove and hereinbelow, the percentages expressed are on a weight basis.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary SO depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the present invention are approximations, the numerical values set forth in the specific examples are reported as precisely as possible. Any numerical value, however, inherently contain certain errors necessarily resulting from the standard deviation found in their respective testing measurements.

Non-limiting examples illustrating the present invention are given below. In the examples, AM means active material.

### EXAMPLE 1

A rinse-out conditioner in accordance with the invention, having the composition below, was prepared:

| Composition | Invention A | B |
|---|---|---|
| Potato starch modified with 2- | 1.5 g | 1.5 g |
| chloroethylaminodipropionic acid neutralized | | |
| with sodium hydroxide (Structure Solanace | | |
| from National Starch) | | |
| Diallyldimethylammonium chloride | 0.5 g AM | |
| homopolymer as an aqueous solution | | |
| comprising 40% AM (Merquat 100 from | | |
| Calgon) | | |
| Amphoteric polymer: | — | 0.5 g AM |
| Terpolymer of diallyldimethylammonium | | |
| chloride, of acrylic acid and of acrylamide as | | |
| an aqueous solution comprising 40% AM | | |
| (Merquat 3300 from Calgon) | | |
| Demineralized water qs | 100.0 g | 100.0 g |

These compositions were applied to washed and drained hair. They were left to stand on the hair for 2 minutes and were then rinsed off with water.

The hair treated with composition A according to the invention was smoother and more supple when wet and had more body and was lighter when dry than the hair treated with composition B.

### EXAMPLE 2

A rinse-out conditioner in accordance with the invention, having the composition below, was prepared:

potato starch modified with 2-chloroethylaminodipropionic acid neutralized with sodium hydroxide (Structure Solanace from National Starch) 1.5 g

combination of myristyl, cetyl and stearyl myristate, palmitate and stearate 0.5 g

amodimethicone sold as a cationic emulsion comprising 35% active material (Fluid DC 939 from Dow Corning) 1.4 g AM

behenyltrimethylammonium chloride as an aqueous solution comprising 80% AM (Genamin KDMP from Clariant) 1.2 g AM

combination of cetyl alcohol and stearyl alcohol (50/50 by weight) 2.5 g

lauryidimethicone copolyol comprising 91% AM (Q2-5200 from Dow Corning) 0.23 g AM

citric acid 0.1 g

fragrance, preserving agents qs

water qs 100 g

The hair treated with the composition according to the invention was smooth and supple when wet and had body and was light when dry.

### EXAMPLE 3

A shampoo in accordance with the invention, having the composition below, was prepared:

| Sodium lauryl ether sulfate (70/30 C12/C14) comprising | 15.5 | g AM |
|---|---|---|
| 2.2 mol of ethylene oxide | | |
| Cocoylbetaine as an aqueous solution comprising 32% | 3 | g AM |
| A.M. | | |
| Hydroxypropyl guar trimethylammonium chloride, sold | 0.1 | g |
| under the name Jaguar C13S by the company Rhodia | | |
| Potato starch modified with 2-chloroethylaminodipropionic | 0.3 | g |
| acid neutralized with sodium hydroxide (Structure Solanace | | |
| from National Starch) | | |
| Polydimethylsiloxane of kinematic viscosity 60,000 cSt | 2.7 | g |
| Amodimethicone as a cationic emulsion comprising 35% | 1.05 | g AM |
| AM (DC939 from Dow Corning) | | |
| 1-(Hexadecyloxy)-2-octadecanol/cetyl alcohol mixture | 2.5 | g |
| Coconut monoisopropanolamide | 0.5 | g |
| Preserving agents, fragrance | qs | |
| Citric acid qs | pH 5.5 | |
| Demineralized water qs | 100 | g |

Shampooing was carried out by applying 12 g of the composition to hair that had been moistened beforehand. The shampoo was worked into a lather and was then rinsed thoroughly with water.

The hair treated with this composition was soft, light and disentangled easily.

### EXAMPLE 4

A rinse-out conditioner in accordance with the invention, having the composition below, was prepared:

| | | |
|---|---|---|
| potato starch modified with 2- | 1 | g |
| chloroethylaminodipropionic acid neutralized with | | |
| sodium hydroxide (Structure Solanace from | | |
| National Starch) | | |
| combination of myristyl, cetyl and stearyl | 0.5 | g |
| myristate, palmitate and stearate | | |
| cationic emulsion comprising 67% AM of | 1.4 | g AM |
| copolymer of polydimethylsiloxane comprising | | |
| a,w-vinyl groups/polydimethylsiloxane | | |
| comprising a,w-hydrogeno groups (DC-1997 | | |
| from Dow Corning) | | |
| behenyltrimethylammonium chloride as an | 1.2 | g AM |
| aqueous solution comprising 80% AM | | |
| (Genamin KDMP from Clariant) | | |
| combination of cetyl alcohol and stearyl | 3 | g |
| alcohol (50/50 by weight) | | |

| | |
|---|---|
| lauryldimethicone copolyol comprising | 91% AM |

| | | |
|---|---|---|
| (Q2-5200 from Dow Corning) | 0.23 | g AM |
| citric acid | 0.1 | g |

| | |
|---|---|
| fragrance, preserving agents | qs |

| | | |
|---|---|---|
| water | qs 100 | g |

### EXAMPLE 5

A rinse-out conditioner in accordance with the invention, having the composition below, was prepared:

| | | |
|---|---|---|
| potato starch modified with 2- | 1 | g |
| chloroethylaminodipropionic acid neutralized | | |
| with sodium hydroxide (Structure Solanace from | | |
| National Starch) | | |
| candelilla wax | 0.3 | g |
| N-oleoyldihydrosphingosine | 0.1 | g |
| trimethylsilyl amodimethicone as a nonionic | 0.92 | g AM |
| emulsion comprising 20% AM | | |
| behenyltrimethylammonium chloride as an | 0.88 | g AM |
| aqueous solution comprising 80% AM | | |
| (Genamin KDMP from Clariant) | | |
| quaternium-87 comprising 75% AM in propylene | 2.5 | g AM |
| glycol (Rewoquat PG 75 from Rewo) | | |
| Stearyl alcohol | 1 | g |
| oxyethylenated sorbitan monolaurate | 0.3 | g |
| quaternized wheat protein hydrolysate | 0.06 | g |

| | |
|---|---|
| fragrance, preserving agents | qs |

| | | |
|---|---|---|
| water | qs 100 | g |

What is claimed is:

1. A cosmetic composition, comprising in a cosmetically acceptable medium:
a) at least one amphoteric starch chosen from the compounds of formulae (I) to (IV): 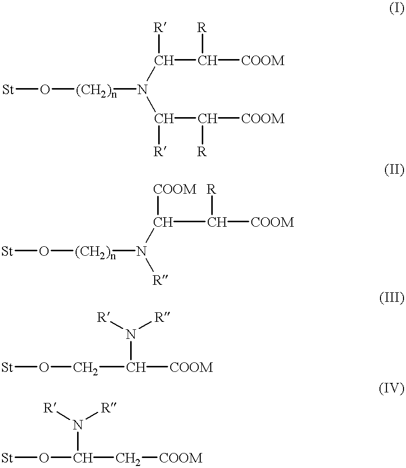
wherein:
St—O is a starch moiety,
R, which may be identical or different, are each chosen from a hydrogen atom and a methyl group,
R′, which may be identical or different, are each chosen from a hydrogen atom, a methyl group, and a —COOH group,
n is chosen from integers ranging from 2 to 3,
M, which may be identical or different, are each chosen from a hydrogen atom, an alkali metal, an alkaline-earth metal, NH₄, quaternary ammonium compounds, and organic amines, and
R″, which may be identical or different, are each chosen from a hydrogen atom, and alkyl groups comprising from 1 to 18 carbon atoms; and
b) at least one cationic conditioner chosen from cationic silicones, quaternary ammonium salt surfactants, cyclopolymers of alkyldiallylamine, cyclopolymers of dialkyldiallylammonium, and polyquaternary ammonium polymers chosen from:
(1) diquaternary ammonium polymers comprising repeating units of formula (IV): 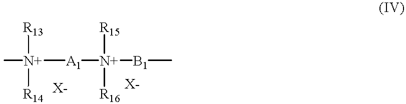
wherein:
R₁₃, R₁₄, R₁₅ and R₁₆, which may be identical or different, are each chosen from aliphatic groups comprising from 1 to 20 carbon atoms, alicyclic groups comprising from 1 to 20 carbon atoms, arylaliphatic groups comprising from 1 to 20 carbon atoms, lower hydroxyalkylaliphatic groups, and, additionally,
at least two of said R₁₃, R₁₄, R₁₅ and R₁₆, with the nitrogen atoms to which they are attached, form at least one heterocycle optionally comprising an additional heteroatom other than nitrogen, and, additionally,
R₁₃, R₁₄, R₁₅ and R₁₆, which may be identical or different, are each chosen from linear and branched C₁-C₆ alkyl groups substituted with at least one group chosen from nitrile groups, ester groups, acyl groups, amide groups and groups chosen from groups of formulae —CO—O—R₁₇—D and —CO—NH—R₁₇—D wherein R₁₇ is chosen from alkylene groups and D is chosen from quaternary ammonium groups;
A₁ and B₁, which may be identical or different, are each chosen from polymethylene groups comprising from 2 to 20 carbon atoms, chosen from linear and branched, saturated and unsaturated polymethylene groups wherein said polymethylene groups may optionally comprise, optionally linked to and optionally intercalated in the main chain, at least one entity chosen from aromatic rings, oxygen atoms, sulfur atoms, sulfoxide groups, sulfone groups, disulfide groups, amino groups, alkylamino groups, hydroxyl groups, quaternary ammonium groups, ureido groups, amide groups and ester groups;
X⁻ is an anion chosen from anions derived from inorganic acids and anions derived from organic acids; and
A₁, R₁₃ and R₁₅ may optionally form, together with the two nitrogen cations to which they are attached, at least one piperazine ring;
with the proviso that if A₁ is chosen from linear and branched, saturated and unsaturated alkylene groups and linear and branched, saturated and unsaturated hydroxyalkylene groups, B₁ may also be chosen from groups of formula:
(CH₂)ₙ—CO—D—OC—(CH₂)ₙ—
wherein D is chosen from:
a) glycol residues of formula: —O—Z—O—, wherein Z is chosen from linear and branched hydrocarbon groups and groups chosen from groups of formulae:
—(CH₂—CH₂—O)ₓ—CH₂—CH₂—;
and
—[CH₂—CH(CH₃)—O]_{y}—CH₂—CH(CH₃)—
wherein x and y, which may be identical or different, are each chosen from integers ranging from 1 to 4 (in which case x and y represent a defined and unique degree of polymerization) and any number ranging from 1 to 4 (in which case x and y represent an average degree of polymerization);
b) bis-secondary diamine residues such as piperazine derivatives;
c) bis-primary diamine residues chosen from residues of formula: —NH—Y—NH—, wherein Y is chosen from linear and branched hydrocarbon groups and residues of formula —CH₂—CH₂—S—S—CH₂—CH₂—; and
d) ureylene groups of formula: —NH—CO—NH—; and
(2) polyquaternary ammonium polymers comprising at least one unit of formula (VII): 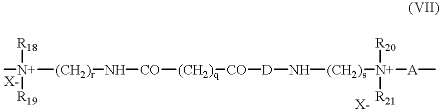
wherein:
R₁₈, R₁₉ R₂₀ and R₂₁, which may be identical or different, are each chosen from a hydrogen atom, a methyl group, an ethyl group, a propyl group, a β-hydroxyethyl group, a β-hydroxypropyl group, and a —CH₂CH₂(OCH₂CH₂)ₚOH group, wherein p is an integer ranging from 0 to 6;
with the proviso that R₁₈, R₁₉, R₂₀ and R₂₁ are all not simultaneously hydrogen atoms;
r and s, which may be identical or different, are each chosen from integers ranging from 1 to 6;
q is an integer ranging from 1 to 34;
X⁻ is chosen from anions of inorganic and organic acids,
D is chosen from direct bonds and —(CH₂)ₜ—CO— groups wherein t is 4 or 7; and
A is chosen from dihalide groups and a group of formula —CH₂—CH₂—O—CH₂—CH₂—.

2. A composition according to claim 1, wherein said at least one amphoteric starch is chosen from the compounds of formulae (I) and (II).

3. A composition according to claim 2, wherein R, R′ and R″ are hydrogen and n is equal to 2.

4. A composition according to claim 1, wherein said cationic silicones are chosen from:
(a) polysiloxanes of formula (VIII): 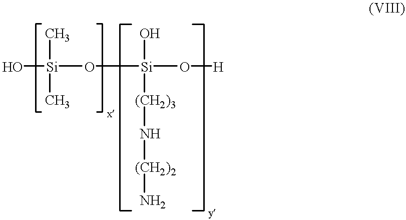
wherein x′ and y′ are chosen from integers dependent on the molecular weight; and
(b) aminosilicones of formula (IX):
R′ₐG₃₋ₐ—Si(OSiG₂)ₙ—(OSiGbR′_{2-b})ₘ—O—SiG₃₋ₐ—R′ₐ (IX)
wherein:
G, which may be identical or different, are each chosen from a hydrogen atom, a phenyl group, an —OH group, and C₁-C₈ alkyl groups,
a, which may be identical or different, are each chosen from integers ranging from 0 to 3,
b is chosen from 0 and 1,
m and n, which may be identical or different, are numbers such that the sum (n+m) ranges from 1 to 2,000, wherein n is chosen from a number ranging from 0 to 1,999, and m is chosen from a number ranging from 1 to 2,000;
R′, which may be identical or different, are each chosen from monovalent groups of formula —C_{q}H_{2q}L, in which q is a number ranging from 2 to 8, and L is an optionally quaternized amine group chosen from the groups:
—NR″—CH₂—CH₂—N′(R″)₂,
—N(R″)₂,
—N^{⊕}(R″)₃A⁻,
—NH^{⊕}(R″)₂A⁻,
—NH₂^{⊕}(R″)A⁻,
and
—N(R″)—CH₂—CH₂—N^{⊕}R″H₂A⁻,
in which:
R″, which may be identical or different, are each chosen from a hydrogen atom, a phenyl group, a benzyl group, and saturated monovalent hydrocarbon groups, and
A⁻ is a halide ion;
(c) aminosilicones of formula (XI): [chemical structure]
wherein:
R₅, which may be identical or different, are each chosen from monovalent hydrocarbon groups comprising 1 to 18 carbon atoms;
R₆ is a divalent hydrocarbon group;
Q⁻ is chosen from anions;
r is an average statistical value ranging from 2 to 20; and
s is an average statistical value ranging from 20 to 200;
d) quaternary ammonium silicones of formula (XII): 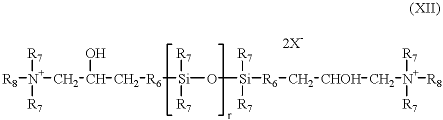
wherein:
R₇, which may be identical or different, are each chosen from monovalent hydrocarbon groups comprising 1 to 18 carbon atoms;
R₆, which may be identical or different, are each chosen from divalent hydrocarbon groups;
R₈, which may be identical or different, are each chosen from a hydrogen atom, and monovalent hydrocarbon groups comprising 1 to 18 carbon atoms;
X⁻ is chosen from anions; and
r is an average statistical value ranging from 2 to 200; and
e) aminosilicones of formula (XIII): 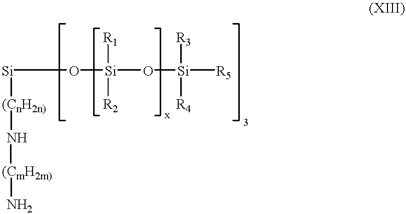
wherein:
R₁, R₂, R₃ and R₄, which may be identical or different, are each chosen from C₁-C₄ alkyl groups, and a phenyl group,
R₅ is chosen from C₁-C₄ alkyl groups, and a hydroxyl group,
n is an integer ranging from 1 to 5,
m is an integer ranging from 1 to 5, and
x is chosen such that the amine number ranges from 0.01 to 1 meq/g.

5. A composition according to claim 4, wherein in said aminosilicones of formula (IX):
a is 0,
b is 1, and
m and n, which may be identical or different, are chosen from numbers such that the sum (n+m) ranges from 50 to 150, wherein n is chosen from a number ranging from 49 to 149, and m is chosen from a number ranging from 1 to 10.

6. A composition according to claim 4, wherein in said aminosilicones of formula (IX), G are each chosen from a methyl group.

7. A composition according to claim 4, wherein in said aminosilicones of formula (IX), R″, which may by identical or different, are each chosen from alkyl groups comprising from 1 to 20 carbon atoms, and A⁻ is an ion chosen from fluoride, chloride, bromide, and iodide ions.

8. A composition according to claim 4, wherein in said aminosilicones of formula (XI), Q⁻ is chosen from halide ions.

9. A composition according to claim 4, wherein in said aminosilicones of formula (XI):
R₅, which may be identical or different, are each chosen from C₁-C₁₈alkyl groups, and C₂-C₁₈ alkenyl groups;
R₆ is a group chosen from C₁-C₁₈ alkylene groups, and divalent C₁-C₁₈ groups;
Q⁻ is chosen from chloride ions and organic acid salts;
r is an average statistical value ranging from 2 to 8; and
s is an average statistical value ranging from 20 to 50.

10. A composition according to claim 4, wherein in said aminosilicones of formula (XI), R₅are each chosen from a methyl group.

11. A composition according to claim 4, wherein in said aminosilicones of formula (XI), R₆ is a C₁-C₈ alkylenoxy group connected to the Si by an SiC bond.

12. A composition according to claim 4, wherein in said aminosilicones of formula (XI), Q⁻ is acetate.

13. A composition according to claim 4, wherein in said quaternary ammonium silicones of formula (XII):
R₇, which may be identical or different, are each chosen from C₁-C₁₈ alkyl groups, C₂-C₁₈ alkenyl groups, and rings comprising 5 to 6 carbon atoms;
R₆, which may be identical or different, are each chosen from C₁-C₁₈ alkylene groups and divalent C₁-C₁₈ groups;
R₈, which may be identical or different, are each chosen from C₁-C₁₈ alkyl groups, C₂-C₁₈ alkenyl groups and groups of formula —R₆—NHCOR₇;
X⁻ is chosen from chloride ions and organic acid salts; and
r is an average statistical value ranging from 5 to 100.

14. A composition according to claim 4, wherein in said quaternary ammonium silicones of formula (XII), R₇ are each chosen from a methyl group.

15. A composition according to claim 4, wherein in said quaternary ammonium silicones of formula (XII), R₆ are each chosen from C₁-C₈ alkylenoxy groups connected to the Si by an SiC bond.

16. A composition according to claim 4, wherein in said quaternary ammonium silicones of formula (XII), X⁻ is acetate.

17. A composition according to claim 4, wherein in said polysiloxanes of formula (VIII), x′ and y′ are chosen from integers such that the weight-average molecular weight of said polysiloxanes of formula (VIII) ranges from 5,000 to 500,000.

18. A composition according to claim 1, wherein said quaternary ammonium salt surfactants are chosen from:
a) quaternary ammonium salts of formula (XIV): 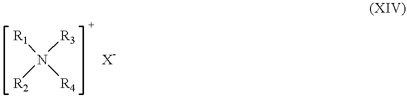
wherein:
X⁻ is an anion chosen from halides, (C₂-C₆)alkyl sulfates, phosphates, alkyl sulfonates, alkylaryl sulfonates, and anions derived from organic acids,
i) the radicals R₁, R₂, and R₃, which may be identical or different, are independently chosen from linear and branched aliphatic radicals comprising from 1 to 4 carbon atoms, optionally comprising hetero atoms and aromatic radicals, and
R₄ is chosen from linear and branched alkyl radicals comprising from 16 to 30 carbon atoms;
ii) the radicals R₁ and R₂, which may be identical or different, are independently chosen from linear and branched aliphatic radicals comprising from 1 to 4 carbon atoms, optionally comprising hetero atoms, and aromatic radicals, and
R₃and R₄, which may be identical or different, are independently chosen from linear and branched alkyl radicals comprising from 12 to 30 carbon atoms, wherein said radicals further comprise at least one function chosen from ester functions and amide functions;
b) quaternary ammonium salts of imidazolinium of formula (XV): 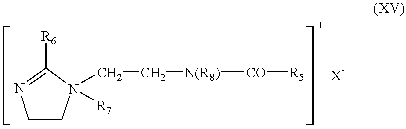
wherein:
R₅ is chosen from alkenyl and alkyl radicals comprising from 8 to 30 carbon atoms,
R₆ is chosen from a hydrogen atom, C₁-C₄ alkyl radicals, alkenyl radicals comprising from 8 to 30 carbon atoms, and alkyl radicals comprising from 8 to 30 carbon atoms,
R₇ is chosen from C₁-C₄ alkyl radicals,
R₈ is chosen from a hydrogen atom and C₁-C₄ alkyl radicals, and
X⁻ is an anion chosen from halides, phosphates, acetates, lactates, alkyl sulfates, alkyl sulfonates, and alkylaryl sulfonates;
c) diquaternary ammonium salts of formula (XVI): 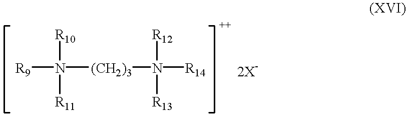
wherein:
R₉ is chosen from aliphatic radicals comprising from 16 to 30 carbon atoms,
R₁₀, R₁₁, R₁₂, R₁₃ and R₁₄, which may be identical or different, are independently chosen from a hydrogen atom and alkyl radicals comprising from 1 to 4 carbon atoms, and
X⁻ is an anion chosen from halides, acetates, phosphates, nitrates and methyl sulfates;
d) quaternary ammonium salts of formula (XVII) comprising at least one ester function: 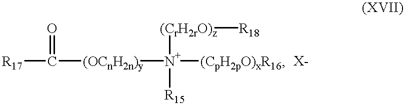
wherein:
R₁₅ is chosen from C₁-C₆ alkyl radicals, C₁-C₆ hydroxyalkyl radicals, and C₁-C₆ dihydroxyalkyl radicals;
R₁₆ is chosen from:
(i) acyl groups of the following formula: 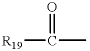
wherein R₁₉ is defined below,
(ii) linear and branched, saturated and unsaturated, C₁-C₂₂ hydrocarbon-based radicals, and
(iii) a hydrogen atom;
R₁₈ is chosen from:
(i) acyl groups of the following formula: 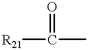
wherein R₂₁ is defined below,
(ii) linear and branched, saturated and unsaturated, C₁-C₆ hydrocarbon-based radicals, and
(iii) a hydrogen atom;
R₁₇, R₁₉ and R₂₁, which may be identical or different, are independently chosen from linear and branched, saturated and unsaturated, C₇-C₂₁ hydrocarbon-based radicals;
n, p and r, which may be identical or different, are independently chosen from integers ranging from 2 to 6;
y is an integer ranging from 1 to 10;
x and z, which may be identical or different, are independently chosen from integers ranging from 0 to 10; and
X⁻ is chosen from simple and complex, organic and inorganic anions;
provided that the sum x+y+z is from 1 to 15, and that when x is 0, then R₁₆ is chosen from linear and branched, saturated and unsaturated, C₁-C₂₂ hydrocarbon-based radicals, and that when z is 0, then R₁₈ is chosen from linear and branched, saturated and unsaturated, C₁-C₆ hydrocarbon-based radicals.

19. A composition according to claim 18, wherein in said quaternary ammonium salts of formula (XVII):
R₁₅ is chosen from a methyl radical and an ethyl radical,
x and y are equal to 1;
z is equal to 0 or 1;
n, p and r are equal to 2;
R₁₆ is chosen from:
(i) acyl groups 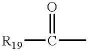
wherein R₁₉ is defined below,
(ii) methyl, ethyl and C₁₄-C₂₂ hydrocarbon-based radicals, and
(iii) a hydrogen atom;
R₁₈ is chosen from:
(i) acyl groups 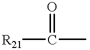
wherein R₂₁ is defined below, and
(iii) a hydrogen atom; and
R₁₇, R₁₉ and R₂₁, which may be identical or different, are independently chosen from linear and branched, saturated and unsaturated, C₁₃-C₁₇ hydrocarbon-based radicals.

20. A composition according to claim 19, wherein R₁₇, R₁₉ and R₂₁ are chosen from linear and branched, saturated and unsaturated C₁₃-C₁₇ aliphatic radicals.

21. A composition according to claim 19, wherein said hydrocarbon-based radicals are chosen from linear hydrocarbon-based radicals.

22. A composition according to claim 18, wherein said quaternary ammonium salts of formula (XVII) are chosen from diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts.

23. A composition according to claim 22, wherein said monoacyloxyethylhydroxyethyldimethylammonium salts are chosen from monoacyloxyethylhydroxyethyldimethylammonium chloride salts and monoacyloxyethylhydroxyethyldimethylammonium methyl sulfate salts.

24. A composition according to claim 18, wherein when R₁₆ and R₁₈ are chosen from acyl groups in said quaternary ammonium salts of formula (XVII), said acyl groups are obtained from plant oils chosen from palm oil and sunflower oil.

25. A composition according to claim 18, wherein X⁻ of said quaternary ammonium salts of formula (XIV) is an anion chosen from chloride, bromide, iodide, methyl sulfate, acetate, and lactate.

26. A composition according to claim 18, wherein said aromatic radicals of said quaternary ammonium salts of formula (XIV) are chosen from aryl and alkylaryl.

27. A composition according to claim 18, wherein said hetero atoms of said quaternary ammonium salts of formula (XIV) are chosen from oxygen, nitrogen, sulfur and halogens.

28. A composition according to claim 18, wherein said aliphatic radicals of said quaternary ammonium salts of formula (XIV) are chosen from alkyl, alkoxy, alkylamide, polyoxy(C₂-C₆)alkylene, and hydroxyalkyl radicals comprising from 1 to 4 carbon atoms.

29. A composition according to claim 18, wherein said R₃ and R₄ of said quaternary ammonium salts of formula (XIV) are chosen from (C₁₂-C₂₂)alkylamido(C₂-C₆)alkyl and (C₁₂-C₂₂)alkylacetate radicals.

30. A composition according to claim 18, wherein in ammonium salts of imidazolinium of formula (XV), said R₅ of formula (XV) is chosen from radicals derived from tallow fatty acid.

31. A composition according to claim 18, wherein in said quaternary ammonium salts of imidazolinium of formula (XV):
R₅ and R₆, which may be identical or different, are independently chosen from alkenyl and alkyl radicals comprising from 12 to 21 carbon atoms,
R₇ is methyl, and
R₈ is hydrogen.

32. A composition according to claim 18, wherein said diquaternary ammonium salts of formula (XVI) comprise propane tallow diammonium dichloride.

33. A composition according to claim 18, wherein said R₁₅ alkyl radicals of said quaternary ammonium salts of formula (XVII) are chosen from linear and branched C₁-C₆ alkyl radicals.

34. A composition according to claim 33, wherein said R₁₅ radicals are linear radicals.

35. A composition according to claim 34, wherein said R₁₅ radicals are chosen from methyl, ethyl, hydroxyethyl and dihydroxypropyl.

36. A composition according to claim 35, wherein said R₁₅ radicals are chosen from methyl and ethyl.

37. A composition according to claim 18, wherein said sum of x+y+z of said quaternary ammonium salts of formula (XVII) ranges from 1 to 10.

38. A composition according to claim 18, wherein said quaternary ammonium salts of formula (XIV) are chosen from (a) compounds comprising at least two fatty aliphatic radicals comprising from 8 to 30 carbon atoms, (b) compounds comprising at least one fatty aliphatic radical comprising from 17 to 30 carbon atoms, and (c) compounds comprising at least one aromatic radical.

39. A composition according to claim 18, wherein said at least one cationic conditioner is chosen from behenyltrimethylammonium salts, stearamidopropyldimethyl(myristyl acetate)ammonium salts, Quaternium-27 and Quaternium-83.

40. A composition according to claim 1, wherein said cyclopolymers of alkyldiallylamine and cyclopolymers of dialkyldiallylammonium are chosen from homopolymers and copolymers comprising at least one unit chosen from units of formulae (XVIII) and (XIX): 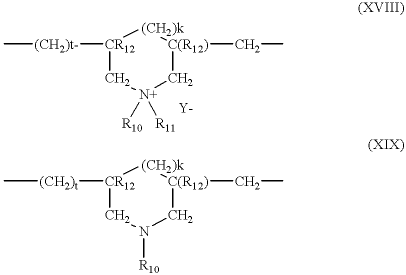
wherein:
k and t, which may be identical or different, are each chosen from 0 and 1, with the proviso that the sum of k+t is equal to 1;
R₁₂, which may be identical or different, are each chosen from a hydrogen atom and a methyl group;
R₁₀ and R₁₁, which may be identical or different, are each chosen from alkyl groups comprising from 1 to 22 carbon atoms, hydroxyalkyl groups wherein the alkyl group optionally comprises from 1 to 5 carbon atoms, lower C₁-C₄ amidoalkyl groups, and, additionally,
R₁₀ and R₁₁, together with the nitrogen atom to which they are commonly attached, form at least one heterocyclic group; and
Y⁻ is an anion.

41. A composition according to claim 40, wherein R₁₀ and R₁₁ are each chosen from piperidyl groups and morpholinyl groups and Y⁻ is chosen from bromide, chloride, acetate, borate, citrate, tartrate, bisulfate, bisulfite, sulfate, and phosphate.

42. A composition according to claim 1, wherein said diquaternary ammonium polymers comprise repeating units of formula (VI): 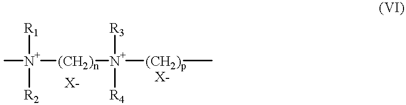
wherein:
R₁, R₂, R₃and R₄, which may be identical or different, are each chosen from alkyl groups comprising from 1 to 4 carbon atoms and hydroxyalkyl groups comprising from 1 to 4 carbon atoms;
n and p, which may be identical or different, are each chosen from integers ranging from 2 to 20; and
X⁻ is an anion chosen from anions derived from inorganic acids and anoins derived from organic acids.

43. A composition according to claim 1, wherein said at least one amphoteric starch is present in an amount ranging from 0.01% to 10% by weight, relative to the total weight of the composition.

44. A composition according to claim 43, wherein said at least one amphoteric starch is present in an amount ranging from 0.1% to 5% by weight, relative to the total weight of the composition.

45. A composition according to claim 1, wherein said at least one cationic conditioner is present in an amount ranging from 0.001% to 10% by weight, relative to the total weight of the composition.

46. A composition according to claim 45, wherein said at least one cationic conditioner is present in an amount ranging from 0.01% to 5% by weight, relative to the total weight of the composition.

47. A composition according to claim 1 further comprising at least one surfactant chosen from anionic, nonionic and amphoteric surfactants.

48. A composition according to claim 47, wherein said at least one surfactant is present in an amount ranging from 0.1% to 60% by weight, relative to the total weight of the composition.

49. A composition according to claim 48, wherein said at least one surfactant is present in an amount ranging from 3% to 40% by weight, relative to the total weight of the composition.

50. A composition according to claim 49, wherein said at least one surfactant is present in an amount ranging from 5% to 30% by weight, relative to the total weight of the composition.

51. A composition according to claim 47, wherein said at least one surfactant is chosen from at least two different surfactants.

52. A composition according to claim 51, wherein said at least two different surfactants are chosen from (a) at least two anionic surfactants, (b) at least one anionic surfactant and at least one amphoteric surfactant, and (c) at least one anionic surfactant and at least one nonionic surfactant.

53. A composition according to claim 1 further comprising at least one additive chosen from thickeners, fragrances, nacreous agents, preserving agents, silicone sunscreens, non-silicone sunscreens, vitamins, provitamins, cationic polymers, amphoteric polymers, anionic polymers, nonionic polymers, proteins, protein hydrolysates, 18-methyleicosanoic acid, hydroxy acids, panthenol, volatile silicones, non-volatile silicones, cyclic silicones, linear silicones, crosslinked silicones, modified silicones, and unmodified silicones.

54. A composition according to claim 53, wherein said at least one additive is present in an amount ranging from greater than 0% to 20% by weight, relative to the total weight of the composition.

55. A composition according to claim 1, wherein said composition has a pH ranging from 2 to 10.

56. A composition according to claim 55, wherein said composition has a pH ranging from 3 to 6.5.

57. A shampoo, a rinse-out conditioner, a leave-in conditioner, a hair permanent-waving composition, a hair straightening composition, a hair dyeing composition, a hair bleaching composition, a rinse-out composition to be applied between steps of a permanent-waving operation, a rinse-out composition to be applied between steps of a hair-straightening operation, comprising, in a cosmetically acceptable medium:
a) at least one amphoteric starch chosen from the compounds of formulae (I) to (IV): 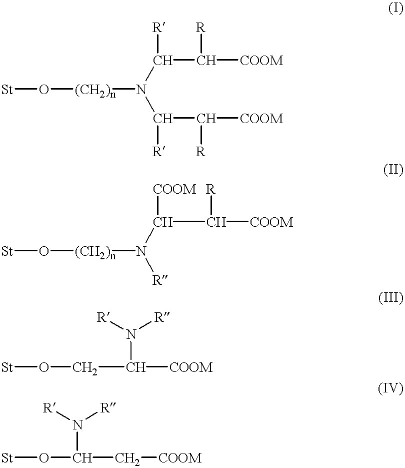
wherein:
St—O is a starch moiety,
R, which may be identical or different, are each chosen from a hydrogen atom and a methyl group,
R′, which may be identical or different, are each chosen from a hydrogen atom, a methyl group, and a —COOH group,
n is chosen from integers ranging from 2 to 3,
M, which may be identical or different, are each chosen from a hydrogen atom, an alkali metal, an alkaline-earth metal, NH₄, quaternary ammonium compounds, and organic amines, and
R″, which may be identical or different, are each chosen from a hydrogen atom, and alkyl groups comprising from 1 to 18 carbon atoms; and
b) at least one cationic conditioner chosen from cationic silicones, quaternary ammonium salt surfactants, cyclopolymers of alkyldiallylamine, cyclopolymers of dialkyldiallylammonium, and polyquaternary ammonium polymers chosen from:
(1) diquaternary ammonium polymers comprising repeating units of formula (IV): 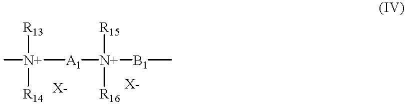
wherein:
R₁₃, R₁₄, R₁₅ and R₁₆, which may be identical or different, are each chosen from aliphatic groups comprising from 1 to 20 carbon atoms, alicyclic groups comprising from 1 to 20 carbon atoms, arylaliphatic groups comprising from 1 to 20 carbon atoms, lower hydroxyalkylaliphatic groups, and, additionally,
at least two of said R₁₃, R₁₄, R₁₅ and R₁₆, with the nitrogen atoms to which they are attached, form at least one heterocycle optionally comprising an additional heteroatom other than nitrogen, and, additionally,
R₁₃, R₁₄, R₁₅ and R₁₆, which may be identical or different, are each chosen from linear and branched C₁-C₆ alkyl groups substituted with at least one group chosen from nitrile groups, ester groups, acyl groups, amide groups and groups chosen from groups of formulae —CO—O—R₁₇—D and —CO—NH—R₁₇—D wherein R₁₇ is chosen from alkylene groups and D is chosen from quaternary ammonium groups;
A₁ and B₁, which may be identical or different, are each chosen from polymethylene groups comprising from 2 to 20 carbon atoms, chosen from linear and branched, saturated and unsaturated polymethylene groups wherein said polymethylene groups may optionally comprise, optionally linked to and optionally intercalated in the main chain, at least one entity chosen from aromatic rings, oxygen atoms, sulfur atoms, sulfoxide groups, sulfone groups, disulfide groups, amino groups, alkylamino groups, hydroxyl groups, quaternary ammonium groups, ureido groups, amide groups and ester groups;
X⁻ is an anion chosen from anions derived from inorganic acids and anions derived from organic acids; and
A₁, R₁₃ and R₁₅ may optionally form, together with the two nitrogen cations to which they are attached, at least one piperazine ring;
with the proviso that if A₁ is chosen from linear and branched, saturated and unsaturated alkylene groups and linear and branched, saturated and unsaturated hydroxyalkylene groups, B₁ may also be chosen from groups of formula:
(CH₂)ₙ—CO—D—OC—(CH₂)ₙ—
wherein D is chosen from:
a) glycol residues of formula: —O—Z—O—, wherein Z is chosen from linear and branched hydrocarbon groups and groups chosen from groups of formulae:
—(CH₂—CH₂—O)ₓ—CH₂—CH₂—; and
—[CH₂—CH(CH₃)—O]_{y}—CH₂—CH(CH₃)—
wherein x and y, which may be identical or different, are each chosen from integers ranging from 1 to 4 (in which case x and y represent a defined and unique degree of polymerization) and any number ranging from 1 to 4 (in which case x and y represent an average degree of polymerization);
b) bis-secondary diamine residues such as piperazine derivatives;
c) bis-primary diamine residues chosen from residues of formula: —NH—Y—NH—, wherein Y is chosen from linear and branched hydrocarbon groups and residues of formula —CH₂—CH₂—S—S—CH₂—CH₂—; and
d) ureylene groups of formula: —NH—CO—NH—; and
(2) polyquaternary ammonium polymers comprising at least one unit of formula (VII): 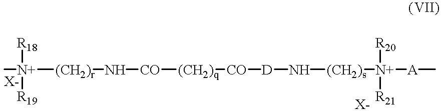
wherein:
R₁₈, R₁₉, R₂₀ and R₂₁, which may be identical or different, are each chosen from a hydrogen atom, a methyl group, an ethyl group, a propyl group, a β-hydroxyethyl group, a β-hydroxypropyl group, and a —CH₂CH₂(OCH₂CH₂)ₚOH group, wherein p is an integer ranging from 0 to 6;
with the proviso that R₁₈, R₁₉, R₂₀ and R₂₁ are all not simultaneously hydrogen atoms;
r and s, which may be identical or different, are each chosen from integers ranging from 1 to 6;
q is an integer ranging from 1 to 34;
X⁻ is chosen from anions of inorganic and organic acids,
D is chosen from direct bonds and —(CH₂)ₜ—CO— groups wherein t is 4 or 7; and
A is chosen from dihalide groups and a group of formula —CH₂—CH₂—O—CH₂—CH₂—.

58. A shower gel, a bubble bath or a make-up-removing product comprising, in a cosmetically acceptable medium:
a) at least one amphoteric starch chosen from the compounds of formulae (I) to (IV): 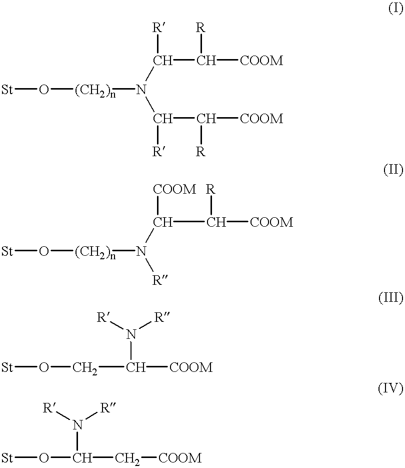
wherein:
St—O is a starch moiety,
R, which may be identical or different, are each chosen from a hydrogen atom and a methyl group,
R′, which may be identical or different, are each chosen from a hydrogen atom, a methyl group, and a —COOH group,
n is chosen from integers ranging from 2 to 3,
M, which may be identical or different, are each chosen from a hydrogen atom, an alkali metal, an alkaline-earth metal, NH₄, quaternary ammonium compounds, and organic amines, and
R″, which may be identical or different, are each chosen from a hydrogen atom, and alkyl groups comprising from 1 to 18 carbon atoms; and
b) at least one cationic conditioner chosen from cationic silicones, quaternary ammonium salt surfactants, cyclopolymers of alkyldiallylamine, cyclopolymers of dialkyldiallylammonium, and polyquaternary ammonium polymers chosen from:
(1) diquaternary ammonium polymers comprising repeating units of formula (IV): 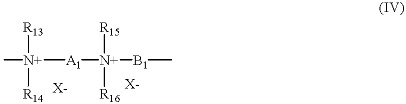
wherein:
R₁₃, R₁₄, R₁₅ and R₁₆, which may be identical or different, are each chosen from aliphatic groups comprising from 1 to 20 carbon atoms, alicyclic groups comprising from 1 to 20 carbon atoms, arylaliphatic groups comprising from 1 to 20 carbon atoms, lower hydroxyalkylaliphatic groups, and, additionally,
at least two of said R₁₃, R₁₄, R₁₅ and R₁₆, with the nitrogen atoms to which they are attached, form at least one heterocycle optionally comprising an additional heteroatom other than nitrogen, and, additionally,
R₁₃, R₁₄, R₁₅ and R₁₆, which may be identical or different, are each chosen from linear and branched C₁-C₆ alkyl groups substituted with at least one group chosen from nitrile groups, ester groups, acyl groups, amide groups and groups chosen from groups of formulae —CO—Q—R₁₇—D and —CO—NH—R₁₇—D wherein R₁₇ is chosen from alkylene groups and D is chosen from quaternary ammonium groups;
A₁ and B₁, which may be identical or different, are each chosen from polymethylene groups comprising from 2 to 20 carbon atoms, chosen from linear and branched, saturated and unsaturated polymethylene groups wherein said polymethylene groups may optionally comprise, optionally linked to and optionally intercalated in the main chain, at least one entity chosen from aromatic rings, oxygen atoms, sulfur atoms, sulfoxide groups, sulfone groups, disulfide groups, amino groups, alkylamino groups, hydroxyl groups, quaternary ammonium groups, ureido groups, amide groups and ester groups;
X⁻ is an anion chosen from anions derived from inorganic acids and anions derived from organic acids; and
A₁, R₁₃ and R₁₅ may optionally form, together with the two nitrogen cations to which they are attached, at least one piperazine ring;
with the proviso that if A₁ is chosen from linear and branched, saturated and unsaturated alkylene groups and linear and branched, saturated and unsaturated hydroxyalkylene groups, B₁ may also be chosen from groups of formula:
(CH₂)ₙ—CO—D—OC—(CH₂)ₙ—
wherein D is chosen from:
a) glycol residues of formula: —O—Z—O—, wherein Z is chosen from linear and branched hydrocarbon groups and groups chosen from groups of formulae:
—(CH₂—CH₂—O)ₓ—CH₂—CH₂—;
and
—[CH₂—CH(CH₃)—O]_{y}—CH₂—CH(CH₃)—
wherein x and y, which may be identical or different, are each chosen from integers ranging from 1 to 4 (in which case x and y represent a defined and unique degree of polymerization) and any number ranging from 1 to 4 (in which case x and y represent an average degree of polymerization);
b) bis-secondary diamine residues such as piperazine derivatives;
c) bis-primary diamine residues chosen from residues of formula: —NH—Y—NH—, wherein Y is chosen from linear and branched hydrocarbon groups and residues of formula —CH₂—CH₂—S—S—CH₂—CH₂—; and
d) ureylene groups of formula: —NH—CO—NH—; and
(2) polyquaternary ammonium polymers comprising at least one unit of formula (VII): 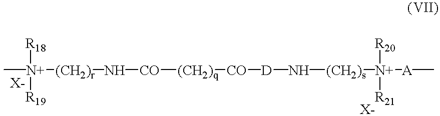
wherein:
R₁₈, R₁₉, R₂₀ and R₂₁, which may be identical or different, are each chosen from a hydrogen atom, a methyl group, an ethyl group, a propyl group, a β-hydroxyethyl group, a β-hydroxypropyl group, and a —CH₂CH₂(OCH₂CH₂)ₚOH group, wherein p is an integer ranging from 0 to 6;
with the proviso that R₁₈, R₁₉, R₂₀ and R₂₁ are all not simultaneously hydrogen atoms;
r and s, which may be identical or different, are each chosen from integers ranging from 1 to 6;
q is an integer ranging from 1 to 34;
X⁻ is chosen from anions of inorganic and organic acids,
D is chosen from direct bonds and —(CH₂)ₜ—Co— groups wherein t is 4 or 7; and
A is chosen from dihalide groups and a group of formula —CH₂—CH₂—O—CH₂—CH₂—.

59. A process for treating a keratin material comprising applying to said keratin material an amount of a composition effective to treat said keratin materials, said composition comprising, in a cosmetically acceptable medium:
a) at least one amphoteric starch chosen from the compounds of formulae (I) to (IV): 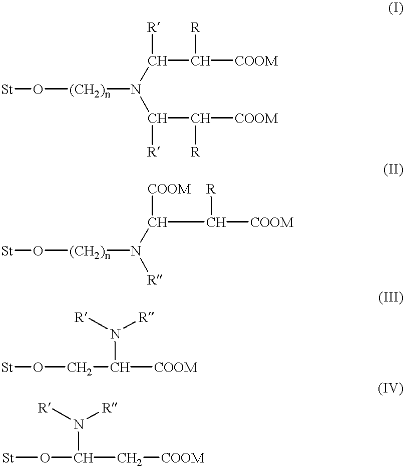
wherein:
St—O is a starch moiety,
R, which may be identical or different, are each chosen from a hydrogen atom and a methyl group,
R′, which may be identical or different, are each chosen from a hydrogen atom, a methyl group, and a —COOH group,
n is chosen from integers ranging from 2 to 3,
M, which may be identical or different, are each chosen from a hydrogen atom, an alkali metal, an alkaline-earth metal, NH₄, quaternary ammonium compounds, and organic amines, and
R″, which may be identical or different, are each chosen from a hydrogen atom, and alkyl groups comprising from 1 to 18 carbon atoms; and
b) at least one cationic conditioner chosen from cationic silicones, quaternary ammonium salt surfactants, cyclopolymers of alkyldiallylamine, cyclopolymers of dialkyldiallylammonium, and polyquaternary ammonium polymers chosen from:
(1) diquaternary ammonium polymers comprising repeating units of formula (IV): 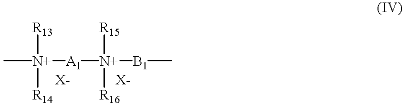
wherein:
R₁₃, R₁₄, R₁₅ and R₁₆, which may be identical or different, are each chosen from aliphatic groups comprising from 1 to 20 carbon atoms, alicyclic groups comprising from 1 to 20 carbon atoms, arylaliphatic groups comprising from 1 to 20 carbon atoms, lower hydroxyalkylaliphatic groups, and, additionally,
at least two of said R₁₃, R₁₄, R₁₅ and R₁₆, with the nitrogen atoms to which they are attached, form at least one heterocycle optionally comprising an additional heteroatom other than nitrogen, and, additionally,
R₁₃, R₁₄, R₁₅ and R₁₆, which may be identical or different, are each chosen from linear and branched C₁-C₆ alkyl groups substituted with at least one group chosen from nitrile groups, ester groups, acyl groups, amide groups and groups chosen from groups of formulae —CO—O—R₁₇—D and —CO—NH—R₁₇—D wherein R₁₇ is chosen from alkylene groups and D is chosen from quaternary ammonium groups;
A₁ and B₁, which may be identical or different, are each chosen from polymethylene groups comprising from 2 to 20 carbon atoms, chosen from linear and branched, saturated and unsaturated polymethylene groups wherein said polymethylene groups may optionally comprise, optionally linked to and optionally intercalated in the main chain, at least one entity chosen from aromatic rings, oxygen atoms, sulfur atoms, sulfoxide groups, sulfone groups, disulfide groups, amino groups, alkylamino groups, hydroxyl groups, quaternary ammonium groups, ureido groups, amide groups and ester groups;
X⁻ is an anion chosen from anions derived from inorganic acids and anions derived from organic acids; and
A₁, R₁₃ and R₁₅ may optionally form, together with the two nitrogen cations to which they are attached, at least one piperazine ring;
with the proviso that if A₁ is chosen from linear and branched, saturated and unsaturated alkylene groups and linear and branched, saturated and unsaturated hydroxyalkylene groups, B₁ may also be chosen from groups of formula:
(CH₂)ₙ—CO—D—OC—(CH₂)ₙ—
wherein D is chosen from:
a) glycol residues of formula: —O—Z—O—, wherein Z is chosen from linear and branched hydrocarbon groups and groups chosen from groups of formulae:
—(CH₂—CH₂—O)ₓ—CH₂—CH₂—;
and
—[CH₂—CH(CH₃)—O]_{y}—CH₂—CH(CH₃)—
wherein x and y, which may be identical or different, are each chosen from integers ranging from 1 to 4 (in which case x and y represent a defined and unique degree of polymerization) and any number ranging from 1 to 4 (in which case x and y represent an average degree of polymerization);
b) bis-secondary diamine residues such as piperazine derivatives;
c) bis-primary diamine residues chosen from residues of formula: —NH—Y—NH—, wherein Y is chosen from linear and branched hydrocarbon groups and residues of formula —CH₂—CH₂—S—S—CH₂—CH₂—; and
d) ureylene groups of formula: —NH—CO—NH—; and
(2) polyquaternary ammonium polymers comprising at least one unit of formula (VII): 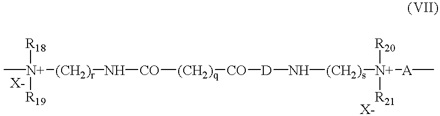
wherein:
wherein x and y, which may be identical or different, are each chosen from integers ranging from 1 to 4 (in which case x and y represent a defined and unique degree of polymerization) and any number ranging from 1 to 4 (in which case x and y represent an average degree of polymerization);
b) bis-secondary diamine residues such as piperazine derivatives;
c) bis-primary diamine residues chosen from residues of formula: —NH—Y—NH—, wherein Y is chosen from linear and branched hydrocarbon groups and residues of formula —CH₂—CH₂—S—S—CH₂—CH₂—; and
d) ureylene groups of formula: —NH—CO—NH—; and
(2) polyquaternary ammonium polymers comprising at least one unit of formula (VII): 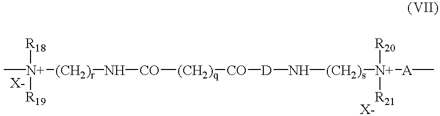
wherein:
R₁₈, R₁₉, R₂₀ and R₂₁, which may be identical or different, are each chosen from a hydrogen atom, a methyl group, an ethyl group, a propyl group, a β-hydroxyethyl group, a β-hydroxypropyl group, and a —CH₂CH₂(OCH₂CH₂)ₚOH group, wherein p is an integer ranging from 0 to 6;
with the proviso that R₁₈, R₁₉, R₂₀ and R₂₁ are all not simultaneously hydrogen atoms;
r and s, which may be identical or different, are each chosen from integers ranging from 1 to 6;
q is an integer ranging from 1 to 34;
X⁻ is chosen from anions of inorganic and organic acids,
D is chosen from direct bonds and —(CH₂)ₜ—CO— groups wherein t is 4 or 7; and
A is chosen from dihalide groups and a group of formula —CH₂—CH₂—O—CH₂—CH₂—.

60. A process according to claim 59, wherein said keratin material is hair.

61. A process according to claim 59, wherein said keratin material is conditioned by the treatment.

62. A process according to claim 59, further comprising rinsing said keratin material with water.

63. A process for washing and conditioning a keratin material comprising applying to said keratin material an effective amount of a composition to wash and condition said keratin material; and rinsing said keratin material with water, said composition comprising, in a cosmetically acceptable medium:
a) at least one amphoteric starch chosen from the compounds of formulae (I) to (IV): 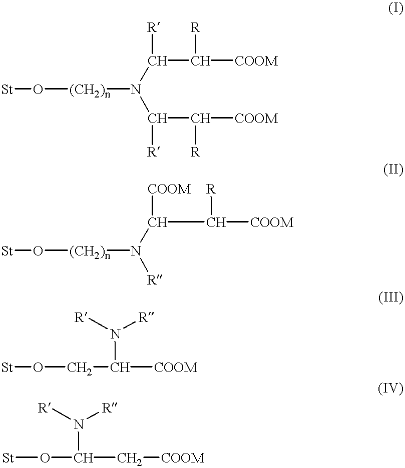
wherein:
St—O is a starch moiety,
R, which may be identical or different, are each chosen from a hydrogen atom and a methyl group,
R′, which may be identical or different, are each chosen from a hydrogen atom, a methyl group, and a —COOH group,
n is chosen from integers ranging from 2 to 3,
M, which may be identical or different, are each chosen from a hydrogen atom, an alkali metal, an alkaline-earth metal, NH₄, quaternary ammonium compounds, and organic amines, and
R″, which may be identical or different, are each chosen from a hydrogen atom, and alkyl groups comprising from 1 to 18 carbon atoms; and
b) at least one cationic conditioner chosen from cationic silicones, quaternary ammonium salt surfactants, cyclopolymers of alkyldiallylamine, cyclopolymers of dialkyldiallylammonium, and polyquaternary ammonium polymers chosen from:
(1) diquaternary ammonium polymers comprising repeating units of formula (IV): 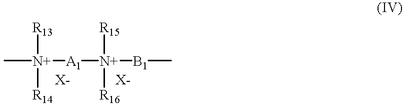
wherein:
R₁₃, R₁₄, R₁₅ and R₁₆, which may be identical or different, are each chosen from aliphatic groups comprising from 1 to 20 carbon atoms, alicyclic groups comprising from 1 to 20 carbon atoms, arylaliphatic groups comprising from 1 to 20 carbon atoms, lower hydroxyalkylaliphatic groups, and, additionally,
at least two of said R₁₃, R₁₄, R₁₅ and R₁₆, with the nitrogen atoms to which they are attached, form at least one heterocycle optionally comprising an additional heteroatom other than nitrogen, and, additionally,
R₁₃, R₁₄, R₁₅ and R₁₆, which may be identical or different, are each chosen from linear and branched C₁-C₆ alkyl groups substituted with at least one group chosen from nitrile groups, ester groups, acyl groups, amide groups and groups chosen from groups of formulae —CO—O—R₁₇—D and —CO—NH—R₁₇—D wherein R₁₇ is chosen from alkylene groups and D is chosen from quaternary ammonium groups;
A₁ and B₁, which may be identical or different, are each chosen from polymethylene groups comprising from 2 to 20 carbon atoms, chosen from linear and branched, saturated and unsaturated polymethylene groups wherein said polymethylene groups may optionally comprise, optionally linked to and optionally intercalated in the main chain, at least one entity chosen from aromatic rings, oxygen atoms, sulfur atoms, sulfoxide groups, sulfone groups, disulfide groups, amino groups, alkylamino groups, hydroxyl groups, quaternary ammonium groups, ureido groups, amide groups and ester groups;
X⁻ is an anion chosen from anions derived from inorganic acids and anions derived from organic acids; and
A₁, R₁₃ and R₁₅ may optionally form, together with the two nitrogen cations to which they are attached, at least one piperazine ring;
with the proviso that if A₁ is chosen from linear and branched, saturated and unsaturated alkylene groups and linear and branched, saturated and unsaturated hydroxyalkylene groups, B₁ may also be chosen from groups of formula:
(CH₂)ₙ—CO—D—OC—(CH₂)ₙ—
wherein D is chosen from:
a) glycol residues of formula: —O—Z—O—, wherein Z is chosen from linear and branched hydrocarbon groups and groups chosen from groups of formulae:
—(CH₂—CH₂—O)ₓ—CH₂—CH₂—;
and
—[CH₂—CH(CH₃)—O]_{y}—CH₂—CH(CH₃)—
wherein x and y, which may be identical or different, are each chosen from integers ranging from 1 to 4 (in which case x and y represent a defined and unique degree of polymerization) and any number ranging from 1 to 4 (in which case x and y represent an average degree of polymerization);
b) bis-secondary diamine residues such as piperazine derivatives;
c) bis-primary diamine residues chosen from residues of formula: —NH—Y—NH—, wherein Y is chosen from linear and branched hydrocarbon groups and residues of formula —CH₂—CH₂—S—S—CH₂—CH₂—; and
d) ureylene groups of formula: —NH—CO—NH—; and
(2) polyquaternary ammonium polymers comprising at least one unit of formula (VII): 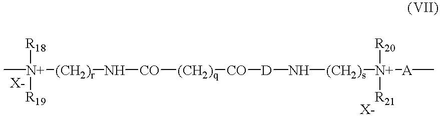
wherein:
R₁₈, R₁₉, R₂₀ and R₂₁, which may be identical or different, are each chosen from a hydrogen atom, a methyl group, an ethyl group, a propyl group, a β-hydroxyethyl group, a β-hydroxypropyl group, and a —CH₂CH₂(OCH₂CH₂)ₚOH group, wherein p is an integer ranging from 0 to 6;
with the proviso that R₁₈, R₁₉, R₂₀ and R₂₁ are all not simultaneously hydrogen atoms;
r and s, which may be identical or different, are each chosen from integers ranging from 1 to 6;
q is an integer ranging from 1 to 34;
X⁻ is chosen from anions of inorganic and organic acids,
D is chosen from direct bonds and —(CH₂)ₜ—CO— groups wherein t is 4 or 7; and
A is chosen from dihalide groups and a group of formula —CH₂—CH₂—O—CH₂—CH₂—.

64. A process according to claim 63, wherein said keratin material is wet before applying said composition.

65. A process according to claim 63, wherein said composition is left to stand on said keratin material for a period of time.

66. A process according to claim 63, wherein said keratin material is hair.
